(19) United States
(12) Patent Application Publication
(10) Pub. No.: US 2001/0046488 A1
(43) Pub. Date: Nov. 29, 2001

(54) COMPOSITIONS AND METHODS FOR DELIVERY OF AN ORGANIZED TISSUE TO AN ORGANISM

(76) Inventors: HERMAN H. VANDENBURGH, PROVIDENCE, RI (US); ROBERT F. VALENTINI, CRANSTON, RI (US)

Correspondence Address: DAVID S. RESNCIK, NIXON PEABODY, LLP, 101 FEDERAL STREET, BOSTON, MA, 02110-1832, US

(21) Appl. No.: 09/342,305

(22) Filed: Jun. 29, 1999

(51) Int. Cl. A61K 48/00; C12N 5/08; C12N 15/00

(52) U.S. Cl. 424/93.21; 424/93.1; 424/93.2; 424/423; 424/424; 435/320.1; 435/325; 435/366; 435/374; 435/383

(57) ABSTRACT

A sleeved organized tissue is formed of a biocompatible structure surrounding organized tissue in at least one dimension and along a length of the tissue In certain preferred embodiments the tissue is attached to the sleeve, subjecting the organized tissue to internal tension within the sleeve. Methods of providing organized tissue within a sleeve and delivering protein to a mammal are also disclosedy

### FIELD OF THE INVENTION

The present invention is directed to delivery of tissue to an organism.

### BACKGROUND

It is known to encapsulate living cells in a biocompatible jacket. It is also known to produce organized tissue. However, nothing in the prior art discloses sleeving an organized tissue construct produced in vitro, forming an organized tissue within a sleeve, nor a sleeve which conforms to the shape of organized tissue contained therein, nor a sleeved organized tissue that is maintained under tension.

It is an object of the present invention to provide sleeved organized tissue which reduces or wholly overcomes some or all of the difficulties inherent in prior known devices. Organized tissue is pre-formed, and unlike cells, can be handled and loaded into a sleeve. A sleeve is particularly important for delivery, deployment and retrieval of the organized tissue. Particular objects and advantages of the invention will be apparent to those skilled in the art, that is, those who are knowledgeable or experienced in this field of technology, in view of the following disclosure of the invention and detailed description of certain preferred embodiments.

### SUMMARY OF THE INVENTION

The principles of the invention may be used to advantage to provide sleeved organized tissue which may be used to deliver protein to a mammal.

In accordance with a first aspect, a sleeved organized tissue is claimed, wherein the sleeve has a biocompatible structure surrounding the organized tissue in at least one dimension and along a length of the tissue.

In accordance with another aspect, a sleeved organized tissue is contemplated wherein the sleeve has a biocompatible preformed structure surrounding organized tissue in at least one dimension and along a length of the tissue.

Thus, the invention encompasses a sleeved organized tissue, wherein the sleeve comprises a biocompatible structure encircling a length of the tissue, or circumferentially surrounding or enclosing the tissue. As used herein, “a length of the tissue” refers to at least 50% of the total length of the tissue, or at least 80%, 90% or even greater than the length of the tissue Where multiple organized tissues are contained within a sleeve, the sleeve will encompass “a length” of at least one such organized tissue, and possibly also of two, three, four or more plural organized tissues The sleeved organized tissue according to the invention also includes a sleeved tissue wherein the tissue is substantially encapsulated or surrounded (i e., encircled along a length, where the length of encirclement is at least 50% of the length of the tissue, or 80%, 90%, or fully encapsulated) by the sleeve.

The invention also contemplates a kit for delivery of a tissue to an organism, the kit comprising a sleeved organized tissue, wherein the sleeve also contains a biocompatible, physiological buffer, and packaging materials therefor, alternatively, the kit may also include a device for delivery of the sleeved organized tissue to the organism, for example, a catheter or syringe and needle, where the sleeved organized tissue is contained within the device or is packaged in the kit separately from the delivery device.

As used herein, “organism” refers to a non-mammal or a mammal, including a human.

In accordance with another aspect, an in vitro method for producing sleeved organized tissue may be performed, wherein the sleeved organized tissue has a biocompatible structure surrounding organized tissue in at least one dimension and along a length of the tissue. This method is performed by providing an organized tissue, placing the organized tissue into a sleeve, wherein the sleeve surrounds the organized tissue in at least one dimension and along a length of the tissue.

In accordance with yet another aspect, an in vitro method for producing sleeved organized tissue having a biocompatible structure surrounding organized tissue in at least one dimension and along a length of the tissue may be performed. This method is performed by providing growing cells, and placing the cells into a sleeve under conditions which permit the cells to form an organized tissue in the sleeve.

In accordance with another aspect, a method of providing a protein to a mammal may be performed. This method is performed by providing a sleeved organized tissue having a biocompatible structure surrounding organized tissue in at least one dimension and along a length of the tissue, wherein the tissue comprises cells which produce a protein, and implanting the sleeved organized tissue into the mammal, wherein the protein is produced in the mammal after the implanting.

In accordance with another aspect, a method of delivering a protein to a mammal may be performed by growing in vitro a plurality of mammalian cells, wherein at least a subset of the cells comprise a foreign DNA sequence operably linked to a promoter and encoding a protein, and wherein the cells are mixed with an extracellular matrix to create a suspension. The suspension is then placed in a vessel wherein the cells form an organized tissue of interest having a three dimensional cellular organization which is retained upon implantation into a mammal The tissue is inserted into a sleeve, and the sleeved tissue is implanted into the mammal, whereby the protein is produced in the mammal and the protein is of a type or produced in an amount not normally produced by cells in the organized tissue.

In accordance with another aspect, a method of delivering a protein to a mammal may be performed by growing in vitro a plurality of mammalian cells, wherein at least a subset of the cells comprises a foreign DNA sequence operably linked to a promoter and encoding a protein, and wherein the cells are mixed with an extracellular matrix to create a suspension. The suspension is then placed in a sleeve, wherein the cells form an organized tissue of interest having a three dimensional cellular organization which is retained upon implantation into a mammal. The sleeved tissue is then implanted into the mammal, whereby the protein is produced in the mammal and the protein is of a type or produced in an amount not normally produced by the cells in the organized tissue.

As used herein with regard to an organized tissue, the term “substantially encapsulated” refers to that which is surrounded or enclosed by- or contained within a sleeve, either on all sides or on all sides except one or both longitudinal termini, or “points for attachment”. Where a sleeve does not fully cover an end of an organized tissue, the sleeve need not physically coincide in length with the organized tissue, but may extend beyond it for a distance as desired.

As used herein with regard to an organized tissue, the terms “longitudinal terminus” or “point for attachment” refer interchangeably to all or a portion of a face of such a tissue seen when the short aspect of an elongated organized tissue is viewed (i.e., when the long axis of the organized tissue is parallel to the sight line of the viewer). As used herein with regard to a longitudinal terminus or point for attachment, the term “portion” refers to as little as 0.001% of such a terminus or point for attachment.

As used herein, “organized tissue” refers to a tissue wherein at least a subset of cells have a cellular organization similar to that of the tissue of origin of those cells (also herein referred to as the “tissue of interest”), and wherein said organized tissue produces a protein of a type- or produced in an amount not produced normally by said tissue of interest in a mammal into which said organized tissue is implanted, comprising: a plurality of cells, wherein at least a subset of said cells comprise a foreign DNA sequence operably linked to a promoter and encoding a protein, wherein said cells form an organized tissue wherein at least a subset of cells of the organized tissue have a cellular organization similar to that of the tissue of origin and wherein the organized tissue further comprises post-mitotic cells.

As used herein, “sleeve” refers to a biocompatible structure, having at least a first point for attachment and a second point for attachment. The sleeve is, in certain preferred embodiments, a porous, preformed structure. The sleeve can have the shape of, for example, a cylinder, a disk, a rectangle, or other suitable geometries The sleeve can also be in the form of a mesh, net, stent or shape-memory material. The sleeve can be constructed from a material selected from the group including, but not limited to, polyacrylates, polymethyl-acrylates, polyalginate, polyvinyl alcohols, polyethylene oxide, polyvinylidene fluoride, polyvinylidenes, polyvinyl chloride, polyurethanes, polyurethane isocyanates, polystyrenes, polyamides, polyaspartate, polyglutamate, cellulose-based polymers, cellulose acetates, cellulose nitrates, polysulfones, polyphosphazenes, polyacrilonitriles, poly(acrilonitrile/covinyl chloride), stretched, woven, extruded or molded polytetrafluoroethylene, stretched, woven, extruded or molded polypropylene, stretched, woven, extruded or molded polyethylene, porous polyvinylidene fluoride, Angel Hair, silicon-oxygen-silicon matrices, polylsine and derivatives, copolymers and mixtures thereof The sleeve can also be constructed of natural materials including, but not limited to, collagen, extracellular matrix, intestinal mucosa, and metals including, but not limited to, stainless steel, tantalum, titanium and its alloys, and nitinol.

As used herein, “sufficiently flexible” refers to that which is capable of undergoing a change in shape, in particular capable of undergoing expansion or retraction, and capable of conforming to the shape of the organized tissue. As used herein, “flexible” does not refer to that which is capable of undergoing a phase change from a liquid to a solid state.

As used herein, “preformed structure” refers to that which has a predetermined solid shape (e.g., porous tube, mesh, or net) and dimensions thereof prior to the insertion of an organized tissue, or prior to the formation of an organized tissue within such a preformed structure.

As used herein “transplantable, substantially encapsulated, organized tissue” refers to a substantially encapsulated organized tissue capable of being implanted into a host mammal.

As used herein, “porous” or refers to having pores, wherein “pore” refers to a small space by which matter can pass through a membrane As used herein with regard to a porous material, the term “selectively permeable” refers to that which allows passage of certain molecules based upon size, surface- or other charge, hydrophilicity/phobicity, topology or other consideration.

As used herein, “retrievable” refers to capable of being recovered. According to the invention, a retrievable, substantially encapsulated, organized tissue can be recovered after implantation into a host mammal in an intact state such that the encapsulated tissue can be reimplanted or the organized tissue can be removed from the sleeve such that the organized tissue maintains its shape after being removed from the sleeve, and the organized tissue can be cultured in vitro under conditions which preserve its in vivo viability after being removed from the sleeve.

As used herein “maintains its shape” refers to an organized tissue which maintains its organized structure after being removed from the sleeve within which it is contained.

As used herein with regard to an organized tissue in a sleeve, “maintains tension” refers to a force of at least 1 pdyne applied by the sleeve to the organized tissue, which force prevents changes in length of the organized tissue of greater than 5% of the starting length of the organized tissue, wherein such tension requires attachment of the first and second points of the organized tissue to first and second points of the sleeve material such that detachment at either point of the tissue from the sleeve results in shortening of the organized tissue or lengthening of the sleeve.

As used herein “retractile forces” refer to forces of at least 1 pdyne that cause an object to contract lengthwise (shorten).

As used herein “permselective” refers to a material having a pore size of approximately 5 to 50 nm. Such a material allows solute exchange at the level of proteins through the pores.

As used herein “microporous” refers to a material having a pore size of approximately 0.5 μm to 10 μm. Such a material allows protein exchange through the pores, but does not allow cell exchange through the pores.

As used herein “macroporous” refers to a material having a pore size of approximately 10 μm to 200 μm. Such a material allows cell passage through the pores as well as vascularization.

As used herein “mesh structure” refers to a material having a pore size of approximately 200 μm to 10 mm Such a material allows direct contact between organized tissue and the host tissue, as well as vascularization. The mesh structure may, in certain preferred embodiments, encompass a large open weave structure.

From the foregoing disclosure, it will be readily apparent to those skilled in the art, that is, those who are knowledgeable or experienced in this area of technology, that the present invention provides a significant technological advance. Preferred embodiments of the sleeved organized tissue of the present invention can provide protein to a mammal using minimally invasive techniques The sleeved organized tissue is implantable in a mammal and due to the structure of the sleeve is identifiable and retrievable. The sleeved tissue can be guided to body tissues and cavities through vascular or non-vascular routes Using, permselective or microporous sleeves will allow the use of allogeneic or xenogeneic cells and tissues These and additional features and advantages of the invention disclosed here will be further understood from the following detailed disclosure of certain preferred embodiments

### BRIEF DESCRIPTION OF THE DRAWINGS

Certain preferred embodiments are described in detail below with reference to the appended drawings wherein:

FIG. 1 is a schematic perspective view of a sleeve of the present invention with organized tissue anchored therein;

FIG. 2 is a schematic perspective view of an alternative embodiment of the sleeved organized tissue of FIG. 1, showing a sleeve having two organized tissues anchored therein;

FIG. 3 is a schematic plan view of an alternative embodiment of the present invention, showing organized tissue anchored to a tension maintaining member; and

FIG. 4 is a graph showing a gluctose-lactase analysis of C2C12-hGH myoblasts encapsulated in PTFE tubes, and

FIG. 5 is a graph showing hGH secretion of C2C12-hGH myoblasts encapsulated in PTFE tubes.

The figures referred to above are not drawn necessarily to scale and should be understood to present a representation of the invention, illustrative of the principles involved. Some features of the sleeved organized tissue depicted in the drawings have been enlarged or distorted relative to others to facilitate explanation and understanding. The same reference numbers are used in the drawings for similar or identical components and features shown in various alternative embodiments Sleeved organized tissue as disclosed herein, will have configurations and components determined, in part, by the intended application and environment in which they are used.

### DETAILED DESCRIPTION OF CERTAIN PREFERRED EMBODIMENTS

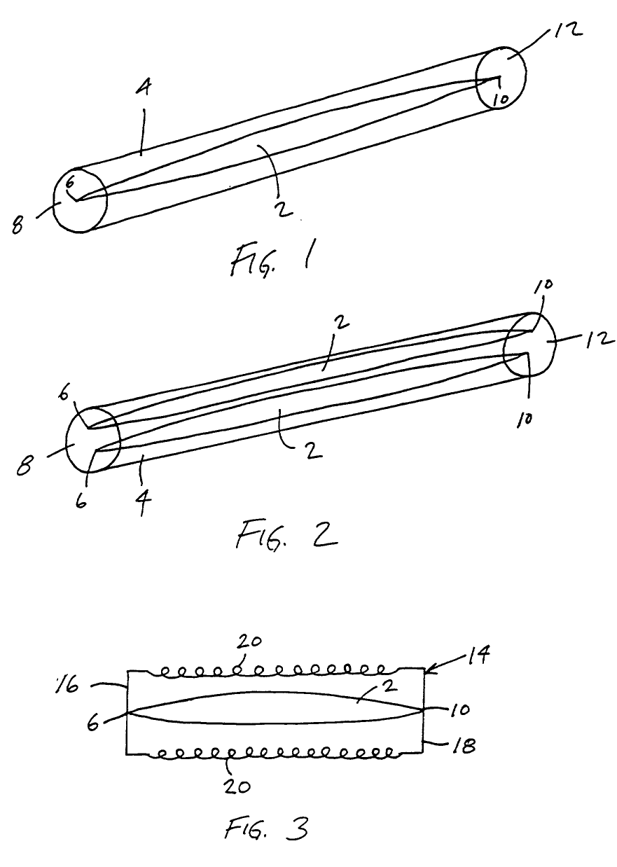

In accordance with a first preferred embodiment as shown in FIG. 1, an organized tissue 2 is positioned within a sleeve 4. Sleeve 4 is a bicompatible structure and, as illustrated in this embodiment, may have a substantially tubular or cylindrical shape. Organized tissue 2 is secured at a first longitudinal terminus, or point for attachment 6 to first end wall 8 of sleeve 4, and at a second longitudinal terminus, or point for attachment 10 to second end wall 12 of sleeve 4. As shown in FIG. 1, sleeve 4 is closed at both ends by end walls 8, 12. Sleeve 4 surrounds the organized tissue in at least one dimension and along a length of the organized tissue.

In certain embodiments, sleeve 4 is sufficiently flexible such that it will conform to the shape of organized tissue 2 Sleeve 4 may be comprised of a shrink wrap material or any suitable material having shape memory which will sufficiently conform to the shape of organized tissue 2.

In certain preferred embodiments, as shown in FIG. 2, a second organized tissue 2 may be positioned within sleeve 4 As shown in FIG. 3, organized tissue 2 may be attached at first point for attachment 6 and second point for attachment 10 to a tension maintaining member 14.

In certain embodiments, sleeve 4 is preferably a preformed structure, having a predetermined shape and dimension prior to insertion of organized tissue therein or prior to the formation of organized tissue therein Sleeve 4 is preferably formed of a porous material, wherein sleeve 4 is selectively permeable in order to allow access of small molecules and proteins while excluding larger molecules. Sleeve 4 may be permselective, having a pore size of approximately 5 to 50 nm and allowing solute exchange at the level of proteins through the pores. Sleeve 4 may be microporous, having a pore size of approximately 0.5 μm to 10 μm and allowing protein exchange through the pores, but not allowing cell exchange through the pores Sleeve 4 may be macroporous, having a pore size of approximately 10 μm to 200 μm and allowing cell passage through the pores as well as vascularization. Sleeve 4 may also have a mesh structure, with a pore size of approximately 200 μm to 10 mm and allowing direct contact between organized tissue and the host tissue, as well as vascularization.

The sleeve can be constructed from a material selected from the group including, but not limited to, polyacrylates, polymethyl-acrylates, polyalginate, polyvinyl alcohols, polyethylene oxide, polyvinylidene fluoride, polyvinylidenes, polyvinyl chloride, polyurethanes, polyurethane isocyanates, polystyrenes, polyamides, polyaspartate, polyglutamate, cellulose-based polymers, cellulose acetates, cellulose nitrates, polysulfones, polyphosphazenes, polyacrilonitriles, poly(acrilonitrile/covinyl chloride), stretched, woven, extruded or molded polytetrafluoroethylene, stretched, woven, extruded or molded polypropylene, stretched, woven, extruded or molded polyethylene, porous polyvinylidene fluoride, Angel Hair, silicon-oxygen-silicon matrices, polylsine and derivatives, copolymers and mixtures thereof The sleeve can also be constructed of natural materials including, but not limited to, collagen, extracellular matrix, intestinal mucosa, and metals including, but not limited to, stainless steel, tantalum, titanium and its alloys, and nitinol

Organized Tissue:

An organized tissue useful in the invention is described in PCT/US97/00303, the contents of which are hereby incorporated by reference. Briefly, the organized tissue has an in vivo-like gross and cellular morphology of a tissue of interest and produces a protein of a type or produced in an amount not produced normally by the tissue of interest comprising a plurality of cells, wherein at least a subset of cells comprise a foreign DNA sequence operably linked to a promoter and encoding a protein, wherein the cells form an organized tissue approximating the in vivo gross morphology of the tissue of interest and wherein the organized tissue is further comprised of post-mitotic cells; and wherein the protein is produced at detectable levels in the tissue.

As used herein, by a “bioactive compound” is meant a compound which influences the biological structure, function, or activity of a cell or tissue of a living organism; for example, a protein.

By “organized tissue” or “organoid” is meant a tissue formed in vitro from a collection of cells having a cellular organization and gross morphology similar to that of the tissue of origin for at least a subset of the cells in the collection An organized tissue or organoid may include a mixture of different cells, for example, muscle (including but not limited to striated muscle, which includes both skeletal and cardiac muscle tissue), fibroblast, and nerve cells, but must exhibit the in vivo cellular organization and gross morphology that is characteristic of a given tissue including at least one of those cells, for example, the organization and morphology of muscle tissue may include parallel arrays of striated muscle tissue.

By “in vivo-like gross and cellular morphology” is meant a three-dimensional shape and cellular organization substantially similar to that of the tissue in vivo.

By “extracellular matrix components” is meant compounds, whether natural or synthetic compounds, which function as substrates for cell attachment and growth Examples of extracellular matrix components include, without limitation, collagen, laminin, fibronectin, vitronectin, elastin, glycosaminoglycans, proteoglycans, and combinations of some or all of these components (e.g, Matrigel™, Collaborative Research, Catalog No. 40234).

An organized tissue useful according to the invention also may be attached to the surface of a substrate via tissue attachment surfaces. By “tissue attachment surfaces” is meant surfaces having a texture, charge or coating to which cells may adhere in vitro. Examples of attachment surfaces include, without limitation, stainless steel wire, VELCRO™, suturing material, titanium, ceramics, native tendon, covalently modified plastics (e.g., RGD complex), and silicon rubber tubing having a textured surface.

By “foreign DNA sequence” is meant a DNA sequence which differs from that of the wild type genomic DNA of the organism and may be extra-chromosomal, integrated into the chromosome, or the result of a mutation in the genomic DNA sequence.

By “substantially post-mitotic cells” is meant an organoid in which at least 50% of the cells containing a foreign DNA sequence are non-proliferative Organoids including substantially post-mitotic cells also may be those in which at least 80%, 90% or even up to 99-100% of the cells containing a foreign DNA sequence are nonproliferative. Cells of an organoid retaining proliferative capacity may include cells of any of the types included in the tissue For example, in striated muscle organoids such as skeletal muscle organoids, the proliferative cells may include muscle stem cells (i.e., satellite cells) and fibroblasts.

I. Production of an Organized Tissue and Transfer to Sleeve

An organized tissue having in vivo-like gross and cellular morphology may be produced in vitro from the individual cells of a tissue of interest As a first step in this process, disaggregated or partially disaggregated cells are mixed with a solution of extracellular matrix components to create a suspension. This suspension is then placed in a vessel having a three dimensional geometry which approximates the in vivo gross morphology of the tissue and includes tissue attachment surfaces coupled to the vessel The cells and extracellular matrix components are then allowed to coalesce or gel within the vessel, and the vessel is placed within a culture chamber and surrounded with media under conditions in which the cells are allowed to form an organized tissue connected to the attachment surfaces.

Although this method is compatible with the in vitro production of a wide variety of tissues, it is particularly suitable for tissues in which at least a subset of the individual cells are exposed to and impacted by mechanical forces during tissue development, remodeling or normal physiologic function. Examples of such tissues include muscle, bone, skin, nerve, tendon, cartilage, connective tissue, endothelial tissue, epithelial tissue, and lung More specific examples include skeletal and cardiac (i.e., striated), and smooth muscle, stratified or lamellar bone, and hyaline cartilage Where the tissue includes a plurality of cell types, the different types of cells may be obtained from the same or different organisms, the same or different donors, and the same or different tissues. Moreover, the cells may be primary cells or immortalized cells. Furthermore, all or some of the cells of the tissue may contain a foreign DNA sequence which mediates the production of a bioactive compound.

The composition of the solution of extracellular matrix components will vary according to the tissue produced. Representative extracellular matrix components include, but are not limited to, collagen, laminin, fibronectin, vitronectin, elastin, glycosaminoglycans, proteoglycans, and combinations of some or all of these components (e.g., Matrigel™, Collaborative Research, Catalog No. 40234). In tissues containing cell types which are responsive to mechanical forces, the solution of extracellular matrix components preferably gels or coalesces such that the cells are exposed to forces associated with the internal tension in the gel.

Culture conditions will also vary according to the tissue produced. Methods for culturing cells are well known in the art and are described, for example, in Skeletal Cell Culture: A Practical Approach, (R I Fveshney, ed IRL Press, 1986). In general, the vessel containing a coalesced suspension of cells and extracellular matrix components is placed in a standard culture chamber (e.g, wells, dishes, or the like), and the chamber is then filled with culture medium until the vessel is submerged The composition of the culture medium is varied, for example, according to the tissue produced, the necessity of controlling the proliferation or differentiation of some or all of the cells in the tissue, the length of the culture period and the requirement for particular constituents to mediate the production of a particular bioactive compound The culture vessel may be constructed from a variety of materials in a variety of shapes as described below.

In the embodiment of the invention wherein the tissue having an in vivo-like gross and cellular morphology is grown in vitro, the vessel in which the tissue is grown also includes tissue attachment surfaces which are an integral part of or coupled to the vessel. Such a vessel may be constructed from a variety of materials which are compatible with the culturing of cells and tissues (e.g., capable of being sterilized and compatible with a particular solution of extracellular matrix components) and which are formable into three dimensional shapes approximating the in vivo gross morphology of a tissue of interest. The tissue attachment surfaces (e.g., stainless steel mesh, VELCRO™, or the like) are coupled to the vessel and positioned such that as the tissue forms in vitro the cells may adhere to and align between the attachment surfaces The tissue attachment surfaces may be constructed from a variety of materials which are compatible with the culturing of cells and tissues (e.g, capable of being sterilized, or having an appropriate surface charge, texture, or coating for cell adherence).

The tissue attachment surfaces may be coupled in a variety of ways to an interior or exterior surface of the vessel or sleeve Alternatively, the tissue attachment surfaces may be coupled to the culture chamber such that they are positioned adjacent the vessel and accessible by the cells during tissue formation In addition to serving as points of adherence, in certain tissue types (e g, muscle), the attachment surfaces allow for the development of tension by the tissue between opposing attachment surfaces. Moreover, where it is desirable to maintain this tension in vivo, the tissue adhered to the tissue attachment surfaces may be transferred to a sleeve according to the invention and the sleeved tissue is then implanted into an organism

A. Production of a Skeletal Muscle Organized Tissue and Transfer to Sleeve

Using the method as generally described above, a skeletal muscle organized tissue having an in vivo-like gross and cellular morphology was produced in vitro During skeletal muscle development embryonic myoblasts proliferate, differentiate, and then fuse to form multi-nucleated myofibers Although the myofibers are nonproliferative, a population of muscle stem cells (i.e., satellite cells), derived from the embryonic myoblast precursor cells, retain their proliferative capacity and serve as a source of myoblasts for muscle regeneration in the adult organism Therefore, either embryonic myoblasts or adult skeletal muscle stem cells may serve as one of the types of precursor cells for in vitro production of a skeletal muscle organoid.

To produce skeletal muscle cells capable of secreting a bioactive compound, primary rat, or avian, or human cells or immortalized murine cells secreting recombinant human growth hormone from a foreign DNA sequence, were suspended in a solution of collagen and Matrigel™ which was maintained at 4° C. to prevent gelling. The cell suspension was then placed in a semi-cylindrical vessel with tissue attachment surfaces coupled to an interior surface at each end of the vessel The vessel was positioned in the bottom of a standard cell culture chamber Following two to four hours of incubation at 37° C., the gelled cell suspension was covered with fresh culture medium (renewed at 24 to 72 hour intervals) and the chamber containing the suspended cells was maintained in a humidified 5% CO₂ incubator at 37° C. throughout the experiment.

Between the first and sixth day of culture, the cells were found to be organized to the extent that they spontaneously detached from the vessel. At this stage, the cells were suspended in culture medium while coupled under tension between tissue attachment surfaces positioned at either end of the culture vessel. During the subsequent ten to fourteen days, the cells formed an organoid containing skeletal myofibers aligned parallel to each other in three dimensions The alignment of the myofibers and the gross and cellular morphology of the organoid were similar to that of in vivo skeletal muscle. To carry out the above method, an apparatus for organoid formation was constructed from silastic tubing and either VELCROTM or metal screens as follows. A section of silastic tubing (approximately 5 mm I.D., 8 mm O.D, and 30 mm length) was split in half with a razor blade and sealed at each end with silicone rubber caulking. Strips of VELCROTM (loop or hook side, 3 mm wide by 4 mm long) or L-shaped strips of stainless steel screen (3 mm wide by 4 mm long by 4 mm high) were then attached with silicone rubber caulking to the interior surface of the split tubing near the sealed ends. The apparatus was thoroughly rinsed with distilled/deionized water and subjected to gas sterilization.

Skeletal muscle organoids were produced in vitro from a C2C12 mouse skeletal muscle myoblast cell line stably co-transfected with recombinant human growth hormone-expressing and β-galactosidase-expressing (β-gal) constructs Dhawan et al., 1991, Science 254:1509-1512 Cells were plated in the vessel at a density of 1-4×10⁶ cells per vessel in 400 μl of a solution containing extracellular matrix components. The suspension of cells and extracellular matrix components was achieved by the following method The solution includes 1 part Matrigel™ (Collaborative Research, Catalog No 40234) and 6 parts of a 1 6 mg/ml solution of rat tail Type I collagen (Collaborative Research, Catalog No 40236) The Matrigel ™ was defrosted slowly on ice and kept chilled until use. The collagen solution was prepared just prior to cell plating by adding to lyophilized collagen, growth medium (see constituents below), and 0.1 N NaOH in volumes equivalent to 90% and 10%, respectively, of the volume required to obtain a final concentration of 1.6 mg/ml and a pH of 7 0-7.3. The collagen, sodium hydroxide and growth medium were maintained on ice prior to and after mixing by inversion.

Freshly centrifuged cells were suspended in the collagen solution by trituration with a chilled sterile pipet. Matrigel™ was subsequently added with a chilled pipet and the suspension was once again mixed by trituration. The suspension of cells and extracellular matrix components was maintained on ice until it was plated in the vessel using chilled pipet tips. The solution was pipetted and spread along the length of the vessel, taking care to integrate the solution into the tissue attachment surfaces The culture chamber containing the vessel was then placed in a standard cell culture incubator, taking care not to shake or disturb the suspension The suspension was allowed to gel, and after 2 hours the culture chamber was filled with growth medium such that the vessel was submerged.

For a period of three days the cells were maintained on growth medium containing DMEM-high glucose (GIBCO-BRL), 5% fetal calf serum (Hyclone Laboratories), and 1% penicillin/streptomycin solution (final concentration 100 units/ml and 0.1 μg/ml, respectively) On the fourth day of culture, the cells were switched to fusion medium containing DMEM-high glucose, 2% horse serum (Hyclone Laboratories), and 100 units/ml penicillin for a period of 4 days On the eighth day of culture, the cells were switched to maintenance medium containing DMEM-high glucose, 10% horse serum, 5% fetal calf serum, and 100 units/ml penicillin for the remainder of the experiment. Before the or organoids were ready for implantation, some were cultured in maintenance media containing 1 μg/ml of cytosine arabinoside for the final four to eight days. Treatment with cytosine arabinoside eliminated proliferating cells and produced organoids including substantially post-mitotic cells.

The cell-extracellular matrix gel (cell-gel) formed in vitro from these stably transfected C2C12 cells reveals cell growth in parallel arrays of highly organized and longitudinally oriented myofibers in mammalian skeletal muscle organoids following three weeks of culturing. Using a pipette, forceps, trocar, suture, or other manipulation, the skeletal muscle organized tissue is then transferred to the sleeve, and the sleeved organized tissue is then ready for transfer to an organism.

II. Production of an Organized Tissue in a Sleeve

An organized tissue may be grown in a sleeve as follows Organized tissue cells in a biocompatible physiological buffered solution are injected in a sleeve having a desired porosity. The sleeve is then placed in a petri dish containing a suitable media solution and maintained under controlled conditions for a number of days. The solution in which the petri dish is maintained may be periodically changed Thus, a kit according to the invention will include a sleeved organized tissue of the invention, comprising a sleeve containing one or more organized tissues, a biocompatible physiological buffered solution in which the organized tissue is maintained within the sleeve for from hours to days to 12 weeks without significant loss of bioactivity, and packaging materials therefor. The biocompatible physiological buffered solution includes, minimally, amino acids, vitamins, essential trace elements and also may include additional components such as growth factors, serum, and tissue extracts.

In Use

In a preferred embodiment, a force is applied by organized tissue 2 to sleeve 4, or vice versa, to maintain tension Thus, organized tissue 2 is longitudinally stretched and/or sleeve 4 is retracted when organized tissue 2 is attached at first and second points for attachment 6, 10, respectively, to sleeve 4. By introducing tension into organized tissue 2, the amount of bioactive compound produced by the tissue can be sustained long-term. Organized tissue 2 may also create retractile forces that reduce the length of sleeve 4.

In certain preferred embodiments, organized tissue 2 may be attached to a tension maintaining member rather than sleeve 4 itself. As shown in FIG. 3, organized tissue 2 may be attached at first point for attachment 6 and second point for attachment 10 to a tension maintaining member 14. In the illustrated embodiment, tension maintaining member 14 comprises first support member 16 and second support member 18 connected to one another by a pair of spring members 20. Organized tissue is anchored at first point for attachment 6 to first support member 16 and at second point for attachment 10 to second support member 18. Tension maintaining member 14 and organized tissue 2 anchored thereto can then be positioned within a sleeve 4. Thus, when organized tissue 2, which is anchored to tension maintaining member 14, is removed from sleeve 4, the organized tissue maintains its shape.

It is to be appreciated that sleeve 4 may, in certain preferred embodiments, be open at first end 8, at second end 12, or at both first end 8 and second end 12.

Sleeve 4, having organized tissue 2 contained therein, may be implanted into a mammal, e.g., a human Sleeve 4 and organized tissue 2 may then be retrieved at a later time from the site of implantation.

In accordance with another preferred embodiment, organized tissue can be produced in vitro by providing organized tissue and placing the organized tissue in a sleeve. The organized tissue may be provided by growing cells and placing the cells in a vessel in which the organized tissue is formed. The organized tissue may then be implanted in a mammal.

In accordance with another preferred embodiment, organized tissue can be produced in vitro by providing growing cells and placing the growing cells into a sleeve under conditions which permit the growing cells to form an organized tissue in the sleeve. The organized tissue is preferably substantially encapsulated within the sleeve. The organized tissue may then be implanted in a mammal.

In accordance with another preferred embodiment, protein may be provided to a mammal, e.g., a human. As a first step in this process, an organized tissue comprising cells which produce a protein is surrounded by a sleeve in at least one dimension and along a length of the organized tissue. The cells may be comprised of like species as the mammal (autologous or allogeneic), or different species (xenogeneic). The sleeved organized tissue is then implanted into a mammal and the protein is produced in the mammal after the implanting The sleeved organized tissue may then be removed from the mammal to terminate delivery of the protein After removal, the organized tissue may be removed from the sleeve and cultured in vitro under conditions which preserve its in vitro viability The organized tissue may then be reinserted into a sleeve, and the sleeved organized tissue may be reimplanted into the mammal to deliver the protein to the mammal Alternatively, after removal, the sleeved organized tissue may be cultured in viro under conditions which preserve its in vivo viability and reimplanted in the mammal.

The organized tissue may be provided by growing a plurality of mammalian cells in vitro, wherein at least a subset of the cells comprise a foreign DNA sequence operably linked to a promoter and encoding the protein, the cells being mixed with an extracellular matrix to create a suspension The suspension may then be placed in a vessel to form an organized tissue of interest having a three dimensional cellular organization which is retained when implanted into a mammal. The tissue may then be inserted into a sleeve.

In accordance with another preferred embodiment, protein may be provided to a mammal, e.g., a human. As a first step in this process, a plurality of mammalian cells are grown in vitro. The cells may be comprised of like species as the mammal At least a subset of the cells comprise a foreign DNA sequence operably linked to a promoter and encoding a protein, and wherein the cells are mixed with an extracellular matrix to create a suspension. The suspension is then placed in a vessel wherein the cells form an organized tissue of interest having a three dimensional cellular organization which is retained upon implantation into a mammal. The organized tissue is then inserted into a sleeve and then implanted into the mammal, whereby the protein is produced in the mammal and the protein is of a type or produced in an amount not normally produced by the cells in the organized tissue.

In accordance with another preferred embodiment, protein may be provided to a mammal, e.g., a human. As a first step in this process, a plurality of mammalian cells are grown in vitro. The cells may be comprised of like species or different species of the mammal. At least a subset of the cells comprise a foreign DNA sequence operably linked to a promoter and encoding a protein, and the cells are mixed with an extracellular matrix to create a suspension. The suspension is then placed in a sleeve, wherein the cells form an organized tissue of interest having a three dimensional cellular organization which is retained upon implantation into a mammal. The sleeved organized tissue is then implanted into the mammal, and the protein is produced in the mammal. The protein is of a type or produced in an amount not normally produced by the cells in the organized tissue.

The sleeved organized tissue may, in certain preferred embodiments, be removed from the mammal to terminate delivery of the protein After removal of the sleeved organized tissue, the organized tissue may be removed from the sleeve and the organized tissue may be cultured in vitro under conditions which preserve its in vivo viability. After culturing, the organized tissue may be reinserted into a sleeve and the sleeved organized tissue may then be reimplanted into the mammal so that the protein is produced in the mammal. The sleeved tissue may be attached to a tether to enhance removal.

Alternatively, after removal of the sleeved organized tissue, the sleeved organized tissue may be cultured in vitro under conditions which preserve its in vivo viability and then reimplanted into the mammal so that the protein is produced in the mammal.

The sleeved organized tissue may be implanted into the tissue of origin of at least one of the cells comprising the organized tissue, or, alternatively, may be implanted into a tissue not of origin of cells comprising the organized tissue.

The protein may be expressed from a foreign DNA sequence comprised of at least a subset of cells of the substantially encapsulated organized tissue. A second protein may be expressed from a second foreign DNA sequence The protein may be growth hormone or a growth factor.

The sleeved organized tissue may comprise skeletal muscle cells, fibroblast cells, or a combination of skeletal muscle cells and fibroblast cells or other cells The sleeved organized tissue may comprise muscle fibers.

Use of Sleeved Organized Tissue to Deliver Bioactive Compound to an Organism

A bioactive compound may be delivered to an organism using a device such as a catheter into which the sleeved organized tissue that produces the bioactive compound has been placed, and after catheterization, implanting the sleeved organized tissue into the organism. Alternatively, the sleeved organized tissue may be directly implanted into the organism using, e.g., surgical forceps, pipette, cannula, trocar, fibrin or other glues, manually or pulling via a suture.

A variety of bioactive compounds may be delivered by this method, and they may function through intracellular (i.e., within the cells of the organized tissue or organoid), endocrine, autocrine, or paracrine mechanisms. Moreover, the organized tissue may deliver multiple bioactive compounds either simultaneously or sequentially (e.g., one bioactive compound mediates the delivery of another) Liberation of the bioactive compound from the cells of the organized tissue may occur by either passive or active processes (e.g., diffusion or secretion).

For example, the bioactive compound may be a hormone, growth factor, or the like which is produced and liberated by the cells of the organized tissue to act locally or systemically on host tissues. Alternatively, the bioactive compound may function within the cells or on the surface of the cells of the organized tissue to enhance the uptake or metabolism of compounds from the host tissue or circulation (e.g., lactic acid, low density lipoprotein) Where the organized tissue serves as a functional and structural adjunct to the host tissue, delivery of growth factors by autocrine or paracrine mechanisms may enhance the integration of the organized tissue into host tissues. Similarly, where multiple bioactive compounds are produced by the organized tissue, autocrine delivery of one of the bioactive compounds may be used to regulate the production of one or more of the other bloactive compounds.

The organized tissue may be implanted at a desired anatomical location within the organism. For example, the organized tissue may be implanted in the same or a different tissue from the tissue of origin of at least one of the individual cells The location of implantation depends, in part, upon the identity of the particular bioactive compound to be delivered. For example, an organized tissue acting as an endocrine organ may be implanted in or adjacent a highly vascularized host tissue Alternatively, an organized tissue acting as a paracrine organ is preferably implanted in or adjacent to the host tissue to which the bioactive compound is to be delivered.

The sleeved organized tissue may be implanted by attachment to a host tissue or as a free floating sleeved organized tissue In addition, attached organized tissues may be implanted with or without the tissue attachment surfaces used for in vitro tissue formation. Tissues responsive to mechanical forces are preferably implanted by attaching directly to the host tissue or by implanting the organized tissue coupled to the attachment surfaces so that the organized tissue is exposed to mechanical forces in vivo. For example, skeletal muscle organized tissue is preferably implanted by attachment to the host tissue under tension along a longitudinal axis of the organized tissue. Moreover, the organized tissue may be permanently or temporarily implanted Permanent implantation may be preferred, for example, where the organized tissue produces a bioactive compound which corrects a systemic metabolic error (e.g, delivery of insulin to treat diabetes), whereas temporary implantation may be preferred where only transient delivery of a bioactive compound is desired (e.g., delivery of a growth factor to enhance wound healing) Furthermore, because organized tissue may be implanted, removed, and maintained in vitro, bloactive compounds may be delivered intermittently to the same or a different location in the organism For example, a skeletal muscle organized tissue produced from the cells of a human patient (e.g, an autograft or allograft) may be implanted at a first anatomical location for a defined period and subsequently implanted at a second location at or after the time of removal.

At least some of the cells of the organized tissue contain a foreign DNA sequence. The foreign DNA sequence may be extra-chromosomal, integrated into the genomic DNA of the organized tissue cell, or may result from a mutation in the genomic DNA of the organized tissue cell In addition, the cells of the organized tissue may contain multiple foreign DNA sequences. Moreover, the different cells of the organized tissue may contain different foreign DNA sequences. For example, in one embodiment, a skeletal muscle organized tissue may include myofibers containing a first foreign DNA sequence and fibroblasts containing a second foreign DNA sequence. Alternatively, the skeletal muscle organized tissue could include myoblasts from different cell lines, each cell line expressing a foreign DNA sequence encoding a different bioactive compound This “mosaic” organized tissue allows the combined and/or synergistic effects of particular bioactive compounds to be exploited. For example, myoblasts expressing growth hormone may be combined with myoblasts expressing an insulin-like growth factor to produce organized tissue useful in stimulating muscle growth/regeneration Similarly, myoblasts expressing a bone morphogenetic protein may be combined with myoblasts expressing a parathyroid hormone to produce organized tissue useful in stimulating bone and cartilage growth/regeneration. A bioactive compound according to the invention can include, but is not limited to, erythropoietin (EPO), insulin-like growth factor-1 (IGF-1), VEGF, β-galactosidase, cytokine, growth hormone, and bone morphogenetic protein.

The foreign DNA sequence may encode a protein which is the bioactive compound. The protein is produced by the cells and liberated from the organized tissue. Alternatively, the DNA sequence may encode an enzyme which mediates the production of a bioactive compound or a cell surface protein which enhances the uptake and metabolism of compounds from the host tissue or circulation (e.g, lactic acid or low density lipoproteins). The DNA sequence may also encode a DNA binding protein which regulates the transcription of the sequence encoding a bioactive compound or an anti-sense RNA which mediates translation of the mRNA for the bioactive compound. The DNA sequence may also bind trans-acting factors such that the transcription of the sequence (i.e, foreign or native) encoding the bioactive compound is enhanced (e.g., by disinhibition) Furthermore, the foreign DNA sequence may be a cis-acting control element such as a promoter or an enhancer coupled to a native or foreign coding sequence for the bioactive compound or for an enzyme which mediates the production of the bioactive compound. Thus, the foreign DNA sequence may be expressible in the cell type into which it is introduced and may encode a protein which is synthesized and which may be secreted by such cells Alternatively, the foreign DNA sequence may be an element that regulates an expressible sequence in the cell.

In order to correlate the delivery dose of an organized tissue implanted in vivo for treatment according to the invention, organized tissue protein secretion levels can be varied by engineering a protein-producing organized tissue with different numbers of protein-secreting myofibers. In addition, varying numbers of organoids can be implanted and levels of bioactive compound determined. For example, where one to four organized tissues producing recombinant human growth hormone (rhGH) has been implanted per animal, and corresponding increase in the level of bioactive compound was found. A correlation was found of in vivo rhGH serum levels from rhGH levels secreted in vitro. A linear relationship was found to exist for the amount of rhGH secreted by rhGH-producing organized tissues preimplantation and postimplantation.

External control of the organized tissue contained within the sleeve is possible Small molecules can pass through pores formed in the sleeve. A porosity size will be selected based on the molecule size of the substance to be passed through the pores For example, antibiotics such as tetracycline, insect steroids, doxycycline, rapomycin, or other molecules may be used which will diffuse across the sleeve to regulate production of a protein from the organized tissue.

In one embodiment of the invention, the pore size of the material which forms the sleeve permits passage of doxycycline (DOX) 1 pg/ml in the culture medium immediately surrounding the sleeve (the organoid is engineered to contain the EPO gene under control of the DOX-activated promoter) After approximately 4 days, DOX-stimulated organoids will secrete approximately 4±0.2μ EPO/day in vitro After introduction into the body, the organoid will maintain the same level of secretion in vivo. Any small molecule gene regulatory system may be used, for example, the gene of interest (e.g., the EPO gene above) may be placed under control of a smallmolecule-sensitive promoter.

The invention is applicable to therapies in which one or more bioactive compounds are delivered to an organism, for example, a mammal, in therapeutically effective levels. A therapeutic gene is one which is expressible in a mammalian, preferably a human, cell and encodes RNA or a polypeptide that is of therapeutic benefit to a mammal, preferably a human. A vector may also include marker genes, such as drug resistance genes, the β-galactosidase gene, the dihydrofolate reductase gene, and the chloramphenicol acetyl transferase gene. A therapeutic effect is evident, for example, where the therapeutic gene encodes a product of physiological importance, such as replacement of a defective gene or an additional potentially beneficial gene function, is expected to confer long term genetic modification of the cells and be effective in the treatment of disease.

The dosages of a bioactive compound administered according to the invention will vary from patient to patient; a “therapeutically effective dose” will be determined by the level of enhancement of function of the transferred genetic material balanced against any risk or deleterious side effects Monitoring levels of gene introduction, gene expression and/or the presence or levels of the encoded product will assist in selecting and adjusting the dosages administered Generally, a composition including a bioactive compound-producing organized tissue according to the invention will be administered in a single dose (per time period in which the organized tissue implant is judged to be effective in producing the bioactive compound), such that the bioactive compound is produced in the mammal in the range of 1 pg-100 mg/kg body weight, preferably in the range of 100 ng-10 μg/kg body weight, depending upon the nature of the bioactive compound, its half-life, and its biological effect By “therapeutically effective amount” also is meant capable of attenuating the clinical symptoms of a disease or a clinical deficiency associated with a disease in an organism by at least 510%, preferably 20-30% and more preferably 35-100%, as compared to an untreated organism.

The compositions and methods of disease treatment according to the invention, is suitable for treating diseases including but not limited to blood disorders, bone and joint disorders, cancer, cardiovascular disorders, endocrine disorders, immune disorders, infectious diseases, wasting disorders, neurological disorders and skin disorders. Treatment of tissue wasting cachexia may be achieved using a bioactive compound which is a growth hormone, insulin and/or insulin-like growth factor, treatment of a neurological disorder may be achieved where the bioactive compound is a nerve growth factor (e.g. NGF, CNTF, or bFGF); treatment of a skin disorder such as a ulcer may be achieved where the bioactive compound is EGF, or wound healing where the bioactive compound is TGF-β or PDGF, treatment of cardiovascular disorders may be achieved where the bioactive compound is vascular endothelial factor or insulin-like growth factor I

### EXAMPLE 1

An experiment was conducted to assess the long-term viability, GH output, and ultrastructure of C2C12 cells transfected with the hGH gene in a microporous, ePTFE tube. The experiment gave the unexpected results that organized tissue grown in a sleeve produced more protein than organized tissue grown in an open trough for the same overall number of cells.

Tube Construction.

Tubes of ePTFE with a pore size of approximately 20 microns were cut into 3 cm segments. On one end of the tube, a stainless steel screw was inserted and loosely tied with 6-0 silk suture. On the other end, a gas-line screw, which has an open channel running from end to end, was inserted and loosely tied with 6-0 silk suture. Upon insertion of both screws, the total open volume within the tube was roughly 0.295 cm³.

Cell Suspension:

C2C12 cells transfected with hGH were grown in Growth Media (20% serum) until confluence in a T-175 flask. The cells were trypsinized and counted. 5.1 million cells were resuspended in a 1.5 mL solution. This solution consisted of 1/7 Matrigel and 6/7 collagel (Type I RTT collagen, NaOH, and C2GM).

Cell Injection:

The cell suspension described above was injected into a total of 4 tubes via a 3.0 cc syringe and 20G1 needle. Each tube was filled with cell suspension containing 1×10⁶ cells, and then the open channel was sealed using Light Cured Resin (manufactured by Ablestik of Califormia) The tube was then placed into a 60 mm petri dish which was filled with 15 mL of C2GM. The dishes were kept in an incubator at 37° C. and 10% CO₂.

Maintenance and Sample Collection:

The media was changed every two days. During the change, 2×1 mL aliquots were saved for GH and glucose-lactase analysis The tubes were kept in GM for the first four days, then FM was used for the next four days, and MM was used for the rest of the study (the tubes were kept for a total of 18 days).

Glucose-Lactase Analysis:

Samples were tested using a YSI Glucose-Lactase Analyzer Model 2000 (manufactured by YSI Incorporated) which provided glucose and lactase concentrations in g/L.

hGH Analysis:

The hGH concentration was assessed using a human growth hormone RIA assay for transient gene expression (from Nichols Institute Diagnostics, Califormia), which gave results in ng/mL.

Results

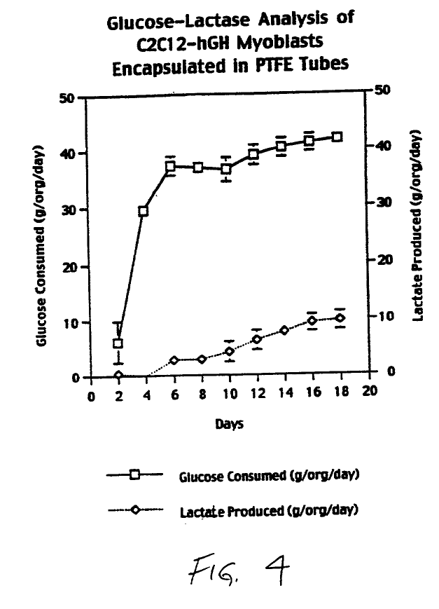

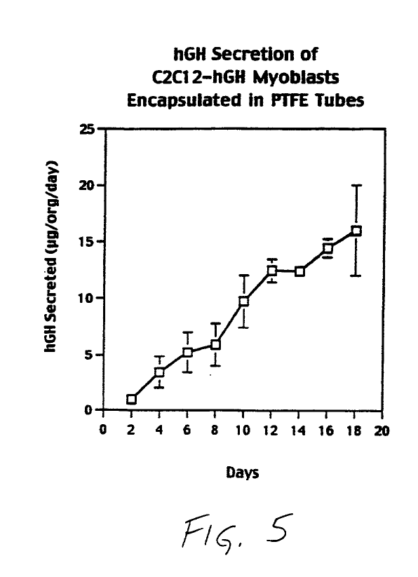

Glucose-lactate analysis is summarized in FIG. 4. Glucose levels rose rapidly through the first four days, and then plateaued thereafter showing cell fusion This is due to the use of Fusion Media (2% serum) from days 4 through 8 to slow proliferation and stimulate myofiber formation via myoblast fusion Growth hormone analysis through day 18, as seen in FIG. 5, indicated that hGH was being released from the organoids into the media. At day 8, the hGH output averaged approximately 5.9 μg hGH/10⁶ cells/day which is similar for 1×10⁶ cells grown in open troughs where output remains at that level In contrast, cells grown in micro-porous tubes showed increasing GH output levels. For example, at day 16 the output rose to roughly 14.4 μg hGH/10⁶ cells/day which is a three fold higher level than for cells grown in open troughs.

In light of the foregoing disclosure of the invention and description of the preferred embodiments, those skilled in this area of technology will readily understand that various modifications and adaptations can be made without departing from the true scope and spirit of the invention All such modifications and adaptations are intended to be covered by the following claims.

1. A sleeved organized tissue, wherein said sleeve comprises a biocompatible structure surrounding said tissue in at least one dimension and along a length of said tissue.

2. The sleeved organized tissue of claim 1, said sleeve having a first point for attachment and a second point for attachment and said organized tissue having a first point for attachment and a second point for attachment, wherein said first point for attachment of said organized tissue is fixed to said first point for attachment of said sleeve.

3. The sleeved organized tissue of claim 2, wherein said second point for attachment of said organized tissue is fixed to said second point for attachment of said sleeve, thereby subjecting said organized tissue to internal tension within said sleeve

4. The sleeved organized tissue of claim 1, wherein said sleeve is sufficiently flexible so as to conform to the shape of the organized tissue.

5. The sleeved organized tissue of claim 1, wherein said sleeved organized tissue is implantable into a mammal

6. The sleeved organized tissue of claim 1, wherein said sleeve comprises a material selected from the group consisting of polyacrylates, polymethyl-acrylates, polyalginate, polyvinyl alcohols, polyethylene oxide, polyvinylidene fluoride, polyvinylidenes, polyvinyl chloride, polyurethanes, polyurethane isocyanates, polystyrenes, polyamides, polyaspartate, polyglutamate, cellulose-based polymers, cellulose acetates, cellulose nitrates, polysulfones, polyphosphazenes, polyacrilonitriles, poly(acrilonitrile/covinyl chloride), stretched or woven, extruded or molded polytetrafluoroethylene, stretched or woven, extruded or molded polypropylene, stretched polyethylene, porous polyvinylidene fluoride, Angel Hair, silicon-oxygensilicon matrices, polylysine, and derivatives, copolymers and mixtures thereof, and metals.

7. The sleeved organized tissue of claim 1, wherein said sleeved organized tissue is retrievable after implantation.

8. The sleeved organized tissue of claim 1, wherein said organized tissue maintains its shape after being removed from the sleeve

9. The sleeved organized tissue of claim 1, wherein said organized tissue within said sleeve Creates retractile forces that reduce the length of the sleeve.

10. The sleeved organized tissue of claim 1, wherein said organized tissue comprises skeletal muscle cells.

11. The sleeved organized tissue of claim 1, wherein said organized tissue comprises fibroblast cells.

12. The sleeved organized tissue of claim 1, wherein said organized tissue comprises skeletal muscle cells and fibroblast cells.

13. The sleeved organized tissue of claim 1, wherein said sleeve is formed of a permselective material.

14. The sleeved organized tissue of claim 13, wherein said permselective material has a pore size of approximately 5 to 50 nm

15. The sleeved organized tissue of claim 1, wherein said sleeve is formed of a microporous material.

16. The sleeved organized tissue of claim 15, wherein said microporous material has a pore size of approximately 0 5 μm to 10 μm

17. The sleeved organized tissue of claim 1, wherein said sleeve is formed of a macroporous material.

18. The sleeved organized tissue of claim 17, wherein said macroporous material has a pore size of approximately 10 μm to 200 μm

19. The sleeved organized tissue of claim 1, wherein said sleeve is formed of a material having a mesh structure.

20. The sleeved organized tissue of claim 19, wherein said mesh structure has a pore size of approximately 200 μm to 10 mm.

21. The sleeved organized tissue of claim 1, wherein said sleeve is open at one end

22. The sleeved organized tissue of claim 1, wherein said sleeve is open at both ends.

23. The sleeved organized tissue of claim 1, wherein said sleeve is closed at both ends.

24. The sleeved organized tissue of claim 1, further comprising a plurality of organized tissues surrounded by said sleeve

25. A sleeved organized tissue, wherein said sleeve comprises a biocompatible preformed structure surrounding said tissue in at least one dimension and along a length of said tissue.

26. An in vitro method for producing the sleeved organized tissue of claim 1, comprising the steps of.
a) providing an organized tissue, and
b) placing said organized tissue into a sleeve, wherein said sleeve surrounds said organized tissue in at least one dimension and along a length of said tissue.

27. The method of claim 26, wherein said providing of said step a) comprises growing cells and placing said cells in a vessel in which said organized tissue is formed

28. The method of claim 26, wherein said method further comprises, after said step b), step c), in which said sleeve organized tissue is implanted into a mammal

29. An in vitro method for producing the sleeved organized tissue of claim 1, comprising the steps of
a) providing growing cells, and
b) placing said cells into a sleeve, under conditions which permit said cells to form an organized tissue in said sleeve.

30. The method of claim 26 or 29 wherein said organized tissue comprises skeletal muscle cells.

31. The method of claim 26 or 29 wherein said organized tissue comprises fibroblast cells.

32. The method of claim 26 or 29 wherein said organized tissue comprises a combination of skeletal muscle cells and fibroblast cells.

33. The method of claim 29, wherein said method further comprises, after said step b), step c), in which said sleeved organized tissue is implanted into a mammal

34. A method of providing a protein to a mammal, comprising the steps of
a) providing a sleeved organized tissue of claim 1, wherein said tissue comprises cells which produce a protein, and
b) implanting into said mammal said sleeved organized tissue, wherein said protein is produced in said mammal after said implanting.

35. The method of claim 34, wherein said providing of said step a) comprises the steps of:
i) growing in vitro a plurality of mammalian cells, wherein at least a subset of said cells comprise a foreign DNA sequence operably linked to a promoter and encoding said protein, and wherein said cells are mixed with an extracellular matrix to create a suspension,
ii) placing said suspension in a vessel wherein the cells form an organized tissue of interest having a three dimensional cellular organization which is retained upon implantation into a mammal, and
iii) inserting said tissue into a sleeve

36. The method of claim 34, wherein said providing of said step a) comprises the steps of:
i) growing in vitro a plurality of mammalian cells, wherein at least a subset of said cells comprise a foreign DNA sequence operably linked to a promoter and encoding said protein, and wherein said cells are mixed with an extracellular matrix to create a suspension; and
ii) placing said suspension in a sleeve, wherein the cells form an organized tissue of interest having a three dimensional cellular organization which is retained upon implantation into a mammal.

37. A method of delivering a protein to a mammal, comprising the steps of
a) growing in vitro a plurality of mammalian cells, wherein at least a subset of said cells comprise a foreign DNA sequence operably linked to a promoter and encoding a protein, and wherein said cells are mixed with an extracellular matrix to create a suspension;
b) placing said suspension in a vessel wherein the cells form an organized tissue of interest having a three dimensional cellular organization which is retained upon implantation into a mammal,
c) inserting said tissue into a sleeve, and
d) implanting said sleeved tissue into said mammal, whereby said protein is produced in said mammal, and whereby said protein is of a type or produced in an amount not normally produced by said organized tissue.

38. A method of delivering a protein to a mammal, comprising the steps of
a) growing in vitro a plurality of mammalian cells, wherein at least a subset of said cells comprise a foreign DNA sequence operably linked to a promoter and encoding a protein, and wherein said cells are mixed with an extracellular matrix to create a suspension;
b) placing said suspension in a sleeve, wherein the cells form an organized tissue of interest having a three dimensional cellular organization which is retained upon implantation into a mammal; and
c) implanting said sleeved tissue into said mammal, whereby said protein is produced in said mammal, whereby said protein is of a type or produced in an amount not normally produced by said organized tissue.

39. The method of claim 34, 37 or 38 further comprising the step of removing said sleeved organized tissue from said mammal to terminate delivery of said protein

40. The method of claim 39, further comprising, following said removal step the steps of:
i) removing said organized tissue from said sleeve, and
ii) culturing said organized tissue in vitro under conditions which preserve its in vivo viability.

41. The method of claim 39 further comprising, following said removal step, the steps of:
i) culturing said organized tissue in vitro under conditions which preserve its in vivo viability; and
ii) reimplanting said organized tissue in said mammal.

42. The method of claim 40, further comprising following said culturing step, the steps of:
A) reinserting said organized tissue into a sleeve; and
B) reimplanting said sleeved organized tissue into said mammal such that said protein is produced in said mammal.

43. The method of claim 34, 37 or 38, wherein said sleeved organized tissue is implanted into the tissue of origin of at least one of said cells comprising said organized tissue.

44. The method of claim 34, 37 or 38, wherein said sleeved organized tissue is implanted into a tissue not of origin of cells comprising said organized tissue.

45. The method of claim 34, 37 or 38, wherein said protein is expressed from a foreign DNA sequence comprised by at least a subset of cells of said sleeved organized tissue.

46. The method of claim 45, wherein in addition to said protein, a second protein is expressed from a second foreign DNA sequence.

47. The method of claim 34, 37 or 38, wherein said protein is a growth factor

48. The method of claim 34, 37 or 38, wherein said protein is growth hormone

49. The method of claim 34, 37 or 38, wherein said sleeved organized tissue comprises skeletal muscle.

50. The method of claim 34, 37 or 38, wherein said tissue comprises muscle fibers.

51. The method of claim 34, 37 or 38, wherein said cells are of like species as said mammal.

52. The method of claim 34, 37 or 38, wherein said mammal is a human

53. A kit for delivery of a tissue to an organism, the kit comprising a sleeved organized tissue, wherein the sleeve contains a biocompatible, physiological buffer, and packaging materials therefor.

54. The kit of claim 53, further comprising a device for delivery of the sleeved organized tissue to the organism

55. The kit of claim 54, wherein said device comprises a catheter.
